SEQUENCE LISTING (1 sequence)

SEQ ID NO: 1 - protein, 240 aa, Vaccinia virus
MAFDISVNASKTINALVYFSTQQNKLVIRNEVNDTHYTVEFDRDKVVDTFISYNRHNDTIEIRGVLPEETNIGCAVNTPVSMTYLYNKYSFKLILAEYIRHRNTISGNIYSALMTLDDLAIKQYGDIDLLFNEKLKVDSDSGLFDFVNFVKDMICCDSRIVVALSSLVSKHWELTNKKYRCMALAEHISDSIPISELSRLRYNLCKYLRGHTESIEDKFDYFEDDDSSTCSAVTDRETDV

(19) United States
(12) Patent Application Publication
(10) Pub. No.: US 2009/0143289 A1
(43) Pub. Date: Jun. 4, 2009

(54) Orthopoxvirus vectors, genes and products thereof

(75) Inventors: Luke Anthony John O'Neill, Dublin (IE); Andrew Graham Bowie, Dublin (IE); Julianne Stack, County Kerry (IE); Geoffrey Lilley Smith, Appleford-on-Thames (GB); Ismar Rocha Haga, Dublin (IE)

Correspondence Address: JACOBSON HOLMAN PLLC, 400 SEVENTH STREET N.W., SUITE 600, WASHINGTON, DC, 20004, US

(73) Assignee: The Provost, Fellows & Scholars of the College of the Holy and Undivided Trinity of Queen Elizabeth

(21) Appl. No.: 12/149,389

(22) Filed: Apr. 30, 2008

Related U.S. Application Data
(63) Continuation of application No. 11/090,057, filed on Mar. 28, 2005, now abandoned.
Application No. 11/090,057 is a continuation of application No. PCT/IE03/00131, filed on Sep. 29, 2003.

(30) Foreign Application Priority Data
Oct. 1, 2002 (IE) 2002/0790

(51) Int. Cl. A61K 31/7088; A61K 38/00

(52) U.S. Cl. 514/12; 514/44; 514/2

(57) ABSTRACT

An orthopoxvirus vector, such as vaccinia, is described in which the A46R protein from vaccinia, or a closely related protein from any orthopoxvirus is not expressed or is expressed but is non-functional. Also described is the use of a vaccinia virus A46R protein or a closely related protein from any orthopoxvirus, or a functional peptide, peptidometic, fragment or derivative thereof, or a DNA vector expressing any of the above in the modulation and/or inhibition of IL1R/TLR superfamily signalling.

### FIELD OF THE INVENTION

The invention relates to a viral protein that is a novel inhibitor of intracellular signalling mediated by the immunologically important interleukin-1/Toll-like receptor (IL-1R/TLR) superfamily. The invention also relates to the mechanism whereby the inhibitor functions, and the use of the inhibitor, or information derived from its mechanism of action, in designing peptides or small molecule inhibitors for use in IL-1R/TLR related diseases and conditions. The invention also relates to a recombinant vaccinia virus (VV) as a vaccine candidate for the prevention of smallpox or other infectious diseases, or for the prevention or treatment of cancer.

### BACKGROUND

Members of the IL-1R/TLR superfamily are key mediators in innate and adaptive immunity (Akira, S., Takeda, K & Kaisho, T. Nature Immunol. 2, 675-680 (2001)). The superfamily is defined by the presence of a cytosolic motif termed the Toll/IL-1 receptor (TIR) domain. The family includes receptors for the proinflammatory cytokines IL-1 and IL-18 as well as the TLR members, which participate in the recognition of pathogens by responding to pathogen associated molecular patterns (PAMPs) and activating signalling pathways leading to altered gene expression (Bowie, A. & O'Neill, L. A. J. J. Leuk. Biol. 67, 508-514 (2000)). The TLRs were discovered on the basis of their amino acid similarity to Toll, a Drosophila protein involved in mediating antifungal defense (Lemaitre, B., et al. Cell 86, 973-983 (1996)). Ten mammalian TLRs have been identified to date. TLR4, the first TLR to be discovered, is essential for the response to lipopolysaccharide (LPS) (Poltorak, A. et al. Science 282, 2085-2088 (1998); Qureshi, S. T. et al. J. Exp. Med. 189, 615-625 (1999)). TLR5 recognises and responds to bacterial flagellin (a 55 kD monomer of bacterial flagella) (Hayashi, F. et al. Nature 410, 1099-1103 (2001)), while TLR9 is required for the recognition of unmethylated CpG motifs which are present in bacterial DNA (Hemmi, H. et al. Nature 408, 740-745 (2000)). TLR2 recognises diverse bacteria and their products, including bacterial lipoproteins, peptidoglycan and other Gram-positive molecular patterns, but only when present as a heterodimer in combination with another TLR, such as TLR1 or TLR6 (Brightbill, H. D. et al. Science 285, 732-736 (1999); Aliprantis, A. et al. Science 285, 736-739 (1999); Underhill, D. et al. Nature 401, 811-815 (1999); Takeuchi, O. et al. Immunity 11, 443-451 (1999); Ozinsky, A. et al. Proc. Natl. Acad. Sci. USA 97, 13766-13771 (2000); Takeuchi, O. et al. Int. Immunol. 13, 933-940 (2001)).

TLRs have also been implicated in sensing viral infections. TLR4 has been shown to be necessary for the cytokine-stimulating ability of F protein from respiratory syncytial virus (RSV) and also for murine retrovirus activation of B cells (Kurt-Jones, E. A. et al. Nature Immunol. 1, 398-401 (2000); Rassa, J. C. et al. Proc. Natl. Acad. Sci. USA 99, 2281-2286 (2002)). TLR3 meanwhile was identified as a receptor activated in response to poly(I:C), a synthetic double-stranded RNA (dsRNA) mimic of viral dsRNA. Poly(I:C) activation of cells via TLR3 led to the activation of the transcription factor NF % B and the production of type I interferons, which are important in anti-viral innate immunity (Alexopoulou, L. et al. Nature 413, 696-712 (2001)). Further, imidazoquinoline compounds known to have potent anti-viral properties, such as R-848, activated immune cells via TLR7 (Hemmi, H. et al. Nature Immunol. 3, 196-200 (2002)).

Since these receptors all contain the signalling TIR domain, stimulation of all the family members with the appropriate ligands leads to activation of NFκB and also the mitogen-activated protein kinases (MAPKs), p38, c-Jun N terminal kinase (JNK) and p42/44. NFκB is a homo- or hetero-dimer of members of the Rel family of transcriptional activators that is involved in the inducible expression of a wide variety of important cellular genes. The activation of NFκB by IL-1, IL-18, TLR2, TLR7 and TLR9 is absolutely dependent on the cytoplasmic TIR domain-containing protein MyD88 (Hemmi, H. et al. Nature Immunol 3, 196-200 (2002); Adachi, O. et al. Immunity, 9, 143-150 (1998); Takeuchi, O. et al. J. Immunol. 164, 554-557 (2000); Schnare, M. et al. Curr. Biol. 10, 1139-1142 (2000)), which is recruited to receptor TIR domains (Medzhitov, R. et al. Mol. Cell 2, 253-258 (1998); Wesche, H. et al. Immunity 7, 837-847 (1997); Muzio, M. et al Science 278, 1612-1615 (1997)). Further, the induction of IL-6 by flagellin via TLR5 was completely dependent on MyD88 (Hayashi, F. et al. Nature 410, 1099-1103 (2001)). TLR4 activates NF % B, by a MyD88-dependent pathway, although an alternative MyD88-independent pathway also exists (Kawai, T. et al. Immunity 11, 115-122 (1999)). Thus MyD88 is a crucial adaptor molecule for the entire IL-1R/TLR superfamily, with the exception of TLR3, where NFκB activation is MyD88-independent (Alexopoulou, L. et al. Nature 413, 696-712 (2001)).

The MyD88 dependent pathway is involved in TNF induction by LPS in dendritic cells whereas the MyD88 independent pathway leads to the upregulation of costimulatory molecules required for dendritic cell maturation, and induction of genes dependent on the transcription factor Interferon Regulatory Factor 3 (IRF3) (Kaisho, T. et al. J. Immunol. 166, 5688-5694 (2001)). An important example of such a gene is Interferon- (IFN). For TLR4 and TLR2, another TIR adapter molecule, MyD88Adaptor-Like (Mal, also known as TIRAP) is involved in the MyD88 dependent pathway (Fitzgerald, K. A. et al. Nature 413, 783 (2001); Horng, T., Barton, G. M. & Medzhitov, R. Nature Immunol. 2, 835-841 (2001); Yamamoto, M. et al Nature 420, 324-329 (2002); Horng, T. et al Nature 420, 329-333 (2002)).

Activation of NFκB by the MyD88 dependent pathway can proceed via recruitment by MyD88 of IL-1 receptor-associated kinase (IRAK) and/or IRAK2, while Mal functions via the recruitment of IRAK2 (Fitzgerald, K. A. et al. Nature 413, 78-83 (2001)). IRAK or IRAK2 activation in turn leads to recruitment of tumour necrosis factor receptor-associated factor 6 (TRAF6). TRAF6 is required for the ubiquitination and activation of the kinase TAK-1, which, in complex with TAB1, phosphorylates IκB kinase (IKK) leading to NFκB activation (Wang, C. et al. Nature 412, 346-351 (2001)).

Other signalling pathways apart from NFκB are also important in TLR-mediated gene induction. Clearly the MAP kinase pathways are also important, leading as they do to the activation of transcription factors, as well as to post-transcriptional events that enhance gene induction. All IL-1R/TLR activators tested have been shown to activate one or all of the three MAP kinase pathways, extracellular regulated kinase (ERK), p38 and JNK. In response to IL-1R/TLR activators, the MAP kinases can be triggered through MyD88 (shown for IL-18, TLR2, TLR9), but also, in the case of LPS and poly(I:C), through MyD88-independent pathways (Adachi, O. et al. Immunity 9, 143-150 (1998); Takeuchi, O. et al. J. Immunol. 164, 554-557 (2000); Hacker, H. et al. J. Exp. Afed 192, 595-600 (2000); Kawai, T. et al. Immunity 11, 115-122 (1999); Alexopoulou, L. et al. Nature 413, 696-712 (2001)). A role for all three MAP kinase pathways in the induction of different genes by LPS has been clearly demonstrated (reviewed in Guha, M. & Mackman, N. Cellular Signalling 13, 85-94 (2001)).

Another important transcription factor activated by some TLR3 and TLR4 is IRF3, which has importance in IFN-dependent anti-viral defense (Servant, M. J., Grandvaux, N. & Hiscott, J. Biochem. Pharmacol. 64, 985-992 (2002)). Recently another TIR adapter termed TICAM-1 or TRIF has been discovered (Yamamoto, M. et al J. Immunol. 169, 6668-6672 (2002); Oshiumi, H. et al Nature Immunol. 4, 161-167 (2003)). It has been shown that for TLR4, TRIF mediates the MyD88-independent pathway to IRF3, while for TLR3, TRIF mediates both NF B and IRF3 activation (Hoebe, K. et al Nature doi:10.1038/nature01889 (2003); Yamamoto, M. et al Science doi:10.1126/science.1087262 (2003)).

Any novel method to inhibit IL-1R/TLR superfamily signalling would have important therapeutic application.

### STATEMENTS OF INVENTION

According to the invention there is provided an orthopoxvirus vector, such as vaccinia, wherein the A46R protein from vaccinia, or a closely related protein from any orthopoxvirus is not expressed or is expressed but is non-functional.

In one embodiment of the invention part or au of the nucleotide sequence encoding A46R is deleted from the viral genome.

In another embodiment of the invention the nucleotide sequence encoding A46R is inactivated by mutation or the insertion of foreign DNA.

The nucleotide sequence encoding A46R may be changed.

In one embodiment of the invention the A46R gene comprises amino acid SEQ ID No. 1.

In another embodiment the vector comprises DNA sequences encoding one or more heterologous polypeptides.

The orthopoxvirus vector of the invention has enhanced immunogenicity and/or safety compared to the wild type orthopoxvirus.

The invention also provides a medicament comprising an orthopoxvirus vector of the invention.

In another aspect the invention provides a vaccine comprising an orthopoxvirus vector of the invention.

In another aspect the invention provides a recombinant orthopoxvirus incapable of expressing a native A46R protein. A vaccine may comprise such a recombinant virus.

In a further aspect the invention provides a method of attenuating an orthopoxvirus vector such as vaccinia virus, comprising the steps of:
(a) deleting part or all of the nucleotide sequence encoding A46R from the viral genome; and/or
(b) inactivating one or more of said nucleotide sequence by mutating said nucleotide sequence or by inserting foreign DNA; and/or
(c) changing said nucleotide sequence to alter the function of a protein product encoded by said nucleotide sequence.

In one embodiment the invention provides a method of inhibiting IL1R/TLR superfamily signalling comprising administering an effective amount of vaccinia A46R protein, or a closely related protein from any orthopoxvirus or a functional peptide, peptidometic, fragment or derivative thereof, or a DNA vector capable of expressing such a protein or fragment thereof.

In another embodiment the invention provides a method of modulating anti-viral immunity in a host comprising administering a vaccinia virus vector of the invention or a functional peptide, peptidometic, fragment or derivative thereof.

The invention also provides an immunogen comprising a vaccinia virus vector or a recombinant virus vector of the invention.

In another aspect the invention provides use of a vaccinia virus A46R protein, or a closely related protein from any orthopoxvirus, or a functional peptide, peptidometic, fragment or derivative thereof, or a DNA vector expressing any of the above in the modulation and/or inhibition of IL-1R/TLR superfamily-signalling.

The use may be in the modulation and/or inhibition of IL-1R/TLR superfamily-induced NFκB activation.

The use may be in the modulation and/or inhibition of IL-1R/TLR superfamily-induced MAP kinase activation.

The use may be in the modulation and/or inhibition of TLR induced IRF3 activation.

In one aspect the vaccinia virus A46R protein, or a closely related protein from any orthopoxvirus, inhibits Toll-like receptor proteins.

The use as may be in the modulation and/or inhibition of NF-κB activity or MAP kinase activation by interaction of A46R with MyD88.

The use may be in the modulation and/or inhibition of NF-κB activity or MAP kinase activation by interaction of A46R with Mal.

In one aspect the vaccinia virus A46R protein inhibits MyD88- and/or Mal-dependent signalling.

The use may be in the modulation and/or inhibition of IRF3 or NF-κB activity by interaction of A46R with TRIF.

In one aspect of the invention the vaccinia virus A46R protein inhibits TRIF-dependent signalling.

The invention also provides a peptide derived from, and/or a small molecule inhibitor designed based on vaccinia virus A46R protein.

The invention further provides a method of screening compounds that modulate the IL-1R/TLR-induced NF-κB or MAP kinase pathway comprising measuring the effect of a test compound on the interaction of vaccinia virus A46R protein or a functional peptide, peptidometic, fragment or derivative thereof with MyD88.

The invention also provides a method of screening compounds that modulate the IL-1R/TLR-induced NF-κB or MAP kinase pathway comprising measuring the effect of a test compound on the interaction of vaccinia virus A46R protein or a functional peptide, peptidometic, fragment or derivative thereof with Mal.

The invention further provides a method of screening compounds that modulate the IL-1R/TLR-induced NF-κB, IRF3 or MAP kinase pathway comprising measuring the effect of a test compound on the interaction of vaccinia virus A46R protein or a functional peptide, peptidometic, fragment or derivative thereof with TRIF.

The invention also provides a method of identifying signalling pathways that require MyD88 and/or Mal and/or TRIF, comprising measuring their sensitivity to A46R.

In another aspect the invention provides use of a functional peptide, peptidometic, or fragment derived from vaccinia virus A46R protein, or a small molecule inhibitor designed based on A46R protein or a DNA vector capable of expressing such a protein or fragment in the treatment and/or prophylaxis of IL-1R/TLR superfamily-induced NF-κB, IRF3 or MAP kinase related diseases or conditions.

Preferably the NF-κB related disease or condition is selected from any one or more of a chronic inflammatory disease, allograft rejection, tissue damage during insult and injury, septic shock and cardiac inflammation, autoimmune disease, cystic fibrosis or any disease involving the blocking of Th1 responses. The chronic inflammatory disease may include any one or more of rheumatoid arthritis, asthma or inflammatory bowel disease. The autoimmune disease may include systemic lupus erythematosus.

The use may be in the treatment and/or prophylaxis of inflammatory disease, infectious disease or cancer.

One aspect of the invention also provides use of a viral protein derived from an A46R-like protein or a functional peptide, gene, or peptidometic thereof in the treatment and/or prophylaxis of inflammatory disease.

The A46R-like protein may be derived from an orthopoxvirus.

The term functional peptide, peptidometic, fragment or derivative as used herein are understood to include any molecule or macromolecule consisting of a portion of the A46R protein, or designed using sequence or structural information from A46R.

The term non-functional is understood to mean not functioning in the normal way compared to how the wild-type A52R protein would function.

The term ‘closely related’ is understood to mean ‘greater than 50% amino acid identity’.

The invention is in the field of poxviruses. The family name is poxvirus, the subfamily name is chordopoxvirinae (infect vertebrates) and the genus is orthopoxvirus which includes species of virus some of which have A46R related proteins. The best known species of this genus are vaccinia, variola, camelpox, cowpox, monkeypox and ectromelia (which infects mice).

The invention relates to any orthopoxvirus vector in which the A46R protein is deleted/modified.

The invention further relates to the use of a DNA vector expressing A46R protein.

### BRIEF DESCRIPTION OF THE DRAWINGS

The invention will be more clearly understood from the following description thereof given by way of example only with reference to the accompanying drawings in which:—

FIG. 1 is a sequence alignment showing the sequence similarity between A46R and TIR domains from IL-1R/TLR signalling proteins;

FIGS. 2a to d are graphs showing the inhibition by A46R of the activation of NFκB and MAP kinases by IL-1 in human 293 cells;

FIGS. 3a to d are graphs showing the inhibition by A46R of the activation of NFκB and MAP kinases by TLR4 in human 293 cells;

FIG. 4 is a graph showing the inhibition by A46R of the activation of NFκB by TLR agonists LPS (TLR4), R-848 (TLR7/8) and flagellin (TLR5) in a murine macrophage cell line, RAW264.7;

FIG. 5 is an immunoblot showing the ectopic expression of A46R in 293T cells;

FIGS. 6a and b are immuno-blots showing association of A46R with MyD88, by co-immunoprecipitation and GST pull-down.

FIGS. 7a and b are immuno-blots showing association of A46R with Mal, by co-immunoprecipitation and GST pull-down.

FIGS. 8a to e are graphs showing the inhibition by A46R of the activation of NFκB and MAP kinases by MyD88 and Mal;

FIGS. 9a and b are bar graphs showing the inhibition by A46R of TLR3-induced Interferon-Stimulated Response Element (ISRE) and Interferon-β (IFNβ) promoter;

FIGS. 10a and b are immuno-blots showing association of A46R with TRIF, by co-immunoprecipitation and GST pull-down.

FIGS. 11a and b are graphs showing the inhibition by A46R of the activation of NFκB and IFNβ promoter by TRIF;

FIGS. 12a to c are graphs showing the inhibition by A46R of TLR4-mediated TRIF-dependent pathways. FIG. 12c also shows inhibition of ISRE by the TLR activators LPS, poly(I:C), R-848 and flagellin;

FIGS. 13a and b are immuno-blots showing the phase during infection at which the A46R protein is expressed, and lack of expression of A46R in cells infected with the VV deletion mutant lacking the A46R gene;

FIGS. 13c and d are graphs showing that deletion of A46R does not affect the growth or replication of VV in cell culture; and

FIG. 14 is a graph showing that deletion of A46R from the vaccinia virus genome attenuates the virus, as measured by weight loss in a murine intranasal model of infection.

### DETAILED DESCRIPTION

Poxviruses are a family of complex DNA viruses that include variola virus, the causative agent of smallpox, and the antigenically related virus used to eradicate this disease, vaccinia virus (VV). Orthopoxvirus such as VV display unique strategies for the evasion of host immune responses such as the ability to produce secreted decoy receptors for cytokines such as IL-1, TNF, and the interferons IFNαβ and IFNγ. Often these inhibitors of host immune function display sequence similarity to host proteins.

The present invention concerns a VV protein A46R, which is known to be an intracellular inhibitor of signalling via IL-1 (Bowie, A. et al. Proc. Natl Acad Sci. USA 97, 10162-10167 (2000)). Using PROFILESEARCH (Genetics Computer Group, Madison, Wis.) to search sequence databases for novel proteins containing TIR domains, we identified A46R (Bowie, A. et al. Proc. Natl Acad Sci. USA 97, 10162-10167 (2000)) as a VV protein with a putative TIR domain. The name A46R is based on the standard VV nomenclature of the Copenhagen strain (Goebel, S. J. et al. Virology 179, 247-266 (1990)). A46R was cloned from the laboratory VV strain Western Reserve (WR), where it was previously called SalF9R (Smith, G. L., Chan, Y. S. & Howard, S. T. J. Gen. Virol. 72, 1349-1376 (1991)). A46R displays a high degree of sequence conservation between different strains of VV, including WR, Copenhagen, Modified Virus Ankara and Tian Tian, while many other orthopoxviruses also have a closely related version of A46R, namely variola major, variola minor, camelpox, monkeypox and cowpox. This high degree of conservation could reflect an important role for A46R in viral virulence.

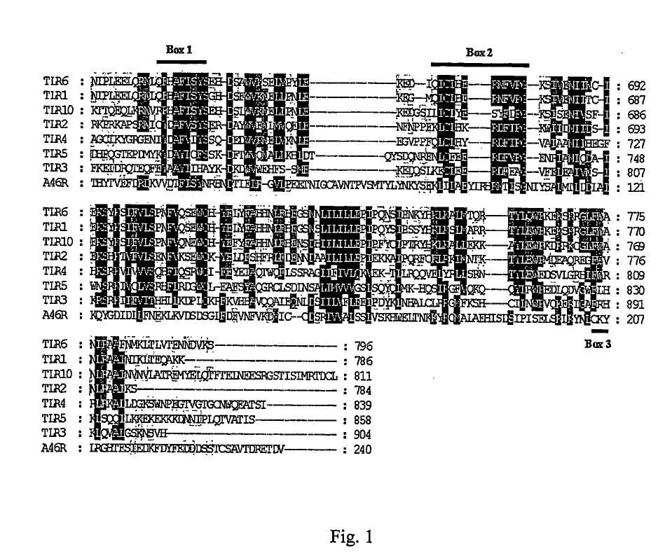

FIG. 1 shows an alignment of A46R with other TIR domains from TLRs. Within the TIR domain there are three regions of important sequence conservation, which have been termed Box 1, 2 and 3 (Bowie, A. & O'Neill, L. A. J. J. Leuk. Biol. 67, 508-514 (2000)). Box 1 is particularly strong in A46R, the sequence DTFISY being as closely related to the Box 1 consensus of other proven family members.

The crystal structures of the TIR domains for TLR1 and TLR2 have been determined and show that the domain adopts a three-layer αβα sandwich conformation, similar to the bacterial protein CheY. Threading the A46R amino acid sequence through secondary structure prediction programmes revealed that A46R could also fold in such a manner. Therefore the A46R protein does appear to contain a bona fide TIR domain.

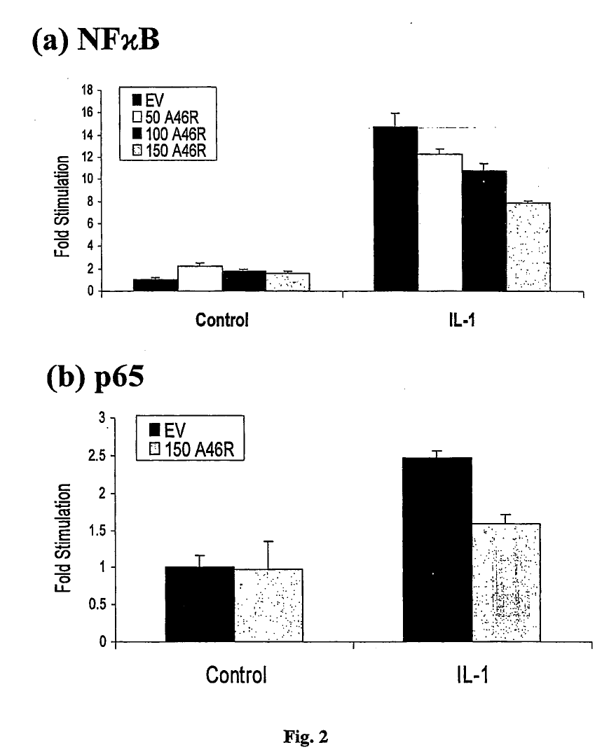

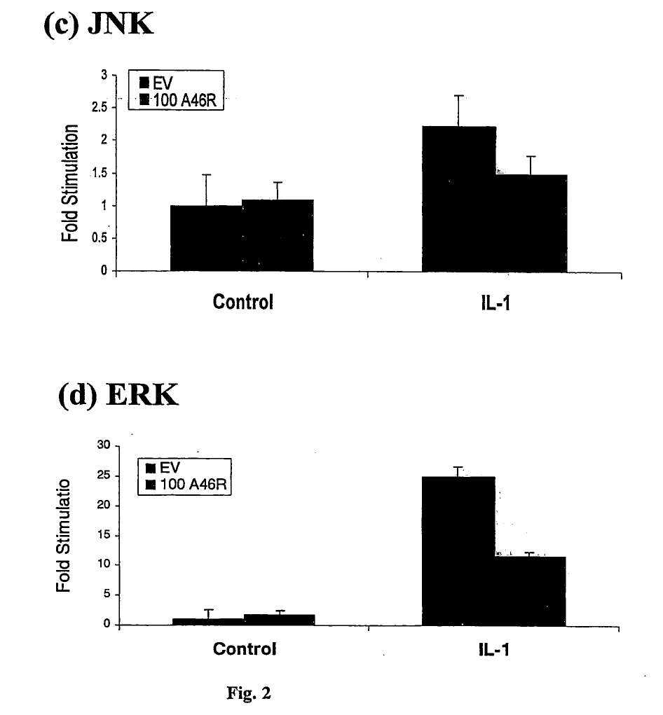

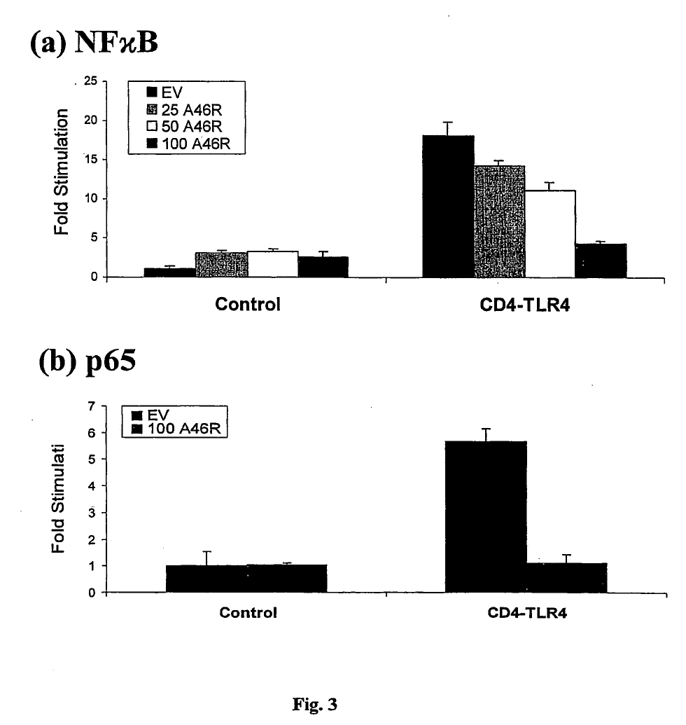

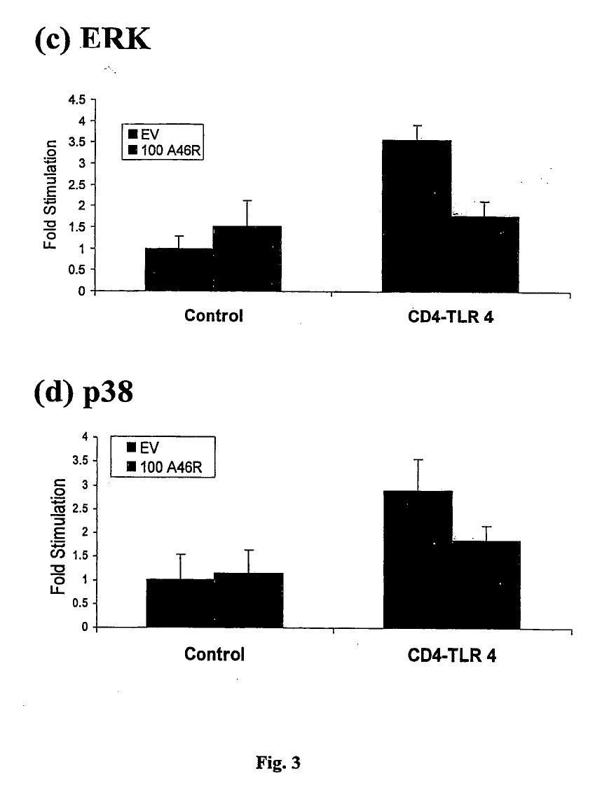

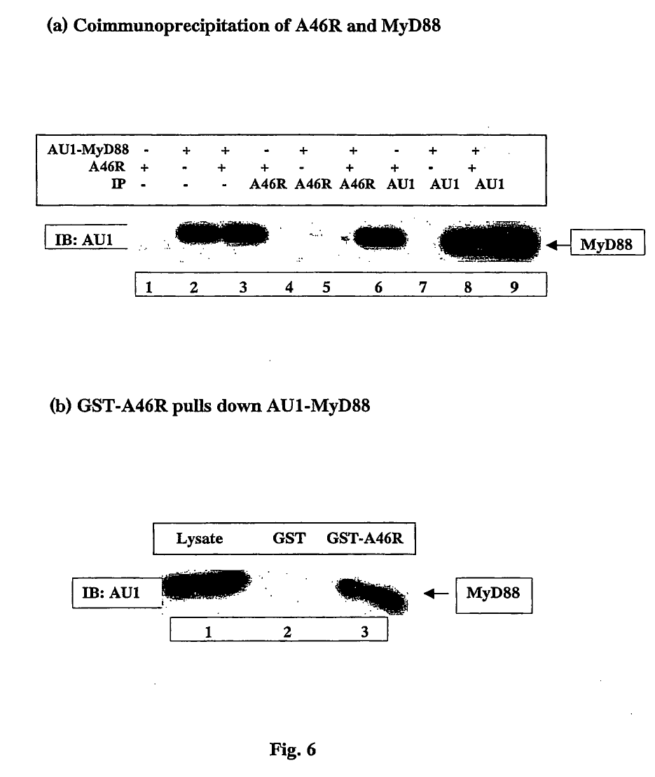

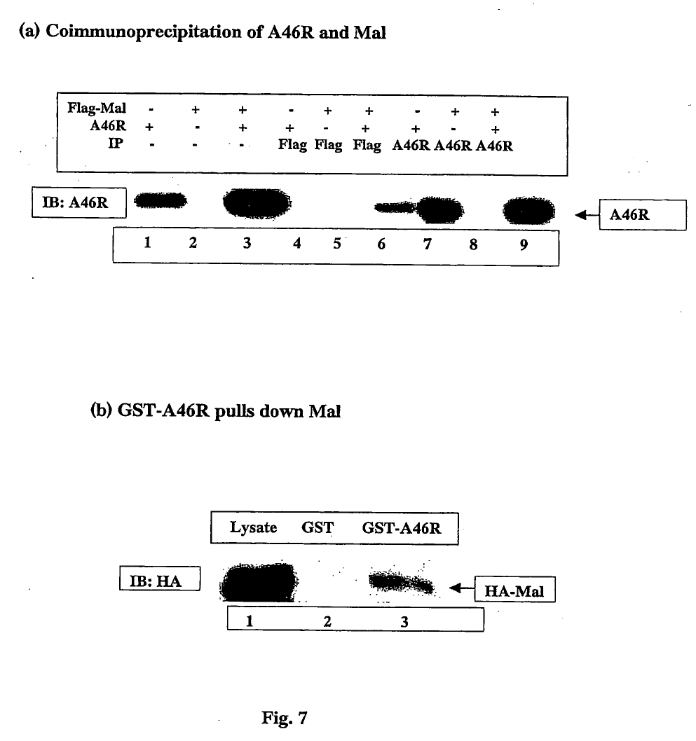

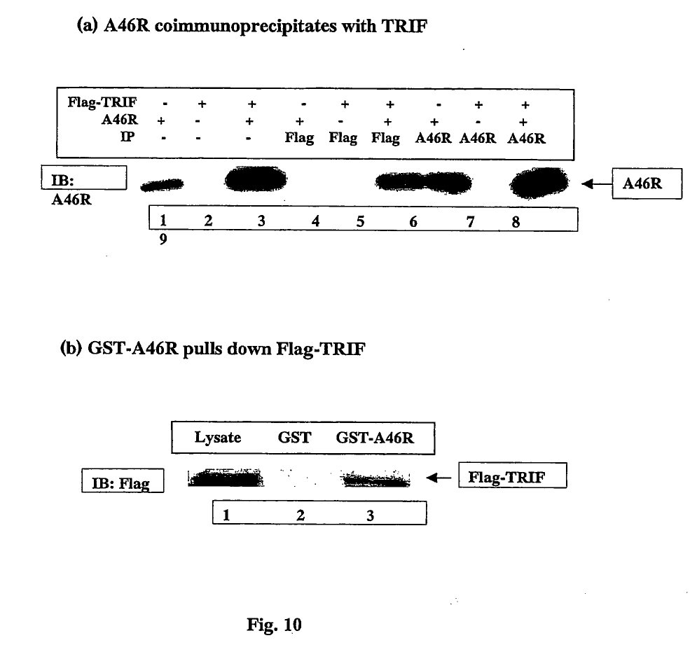

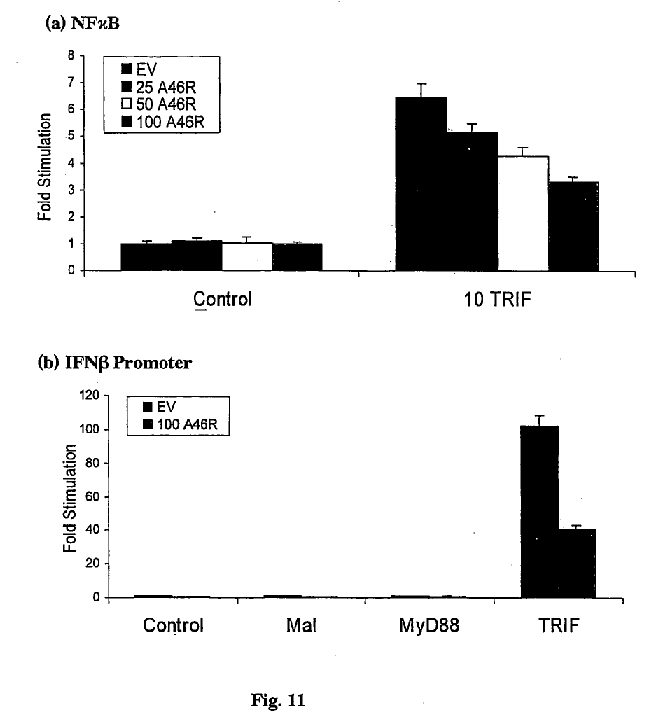

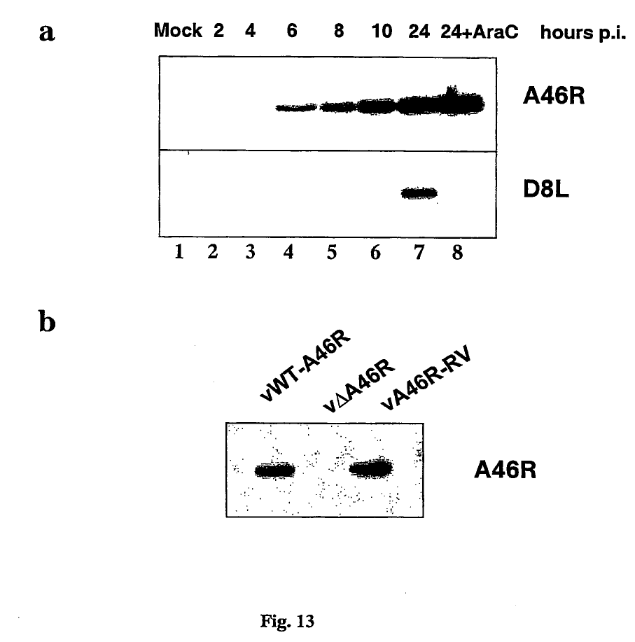

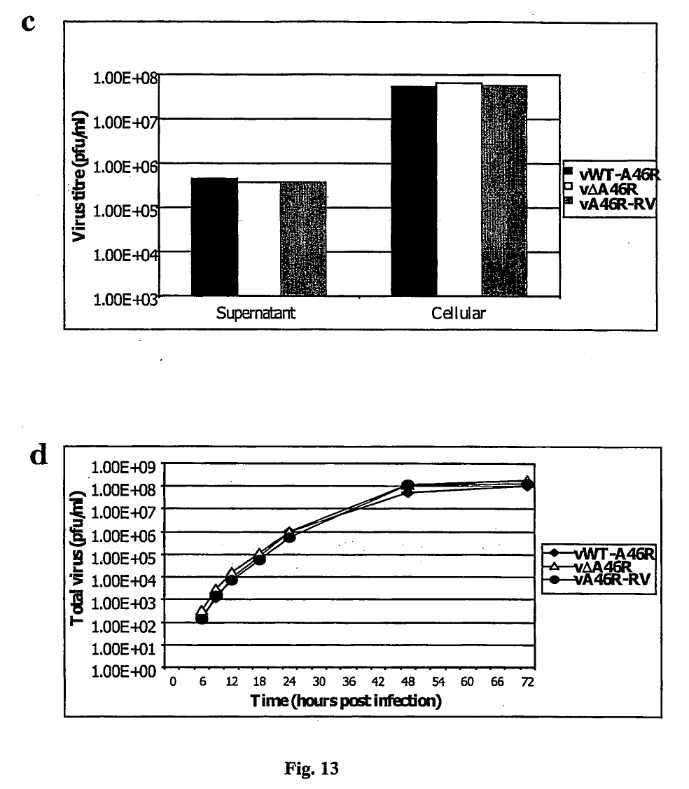

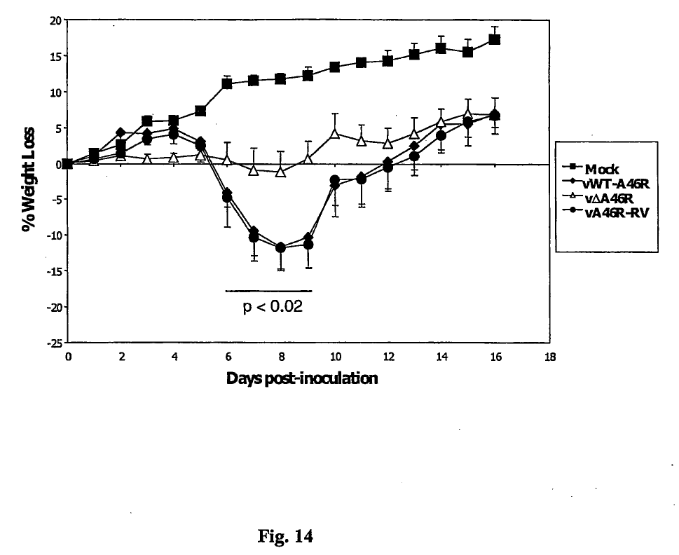

In the present invention it was found that, in addition to blocking IL-1 mediated NFκB activation A46R inhibits numerous signalling pathways activated by IL-1, including c-Jun N terminal kinase (JNK) and extracellular-regulated kinase (ERK) MAP kinase activation (FIG. 2a to d). Further, A46R is also shown to inhibit NFκB activation by multiple TLRs including TLR4 (FIG. 3a), TLR7/8 (FIG. 4) and TLR5 (FIG. 4). The fact that A46R could associate with both MyD88 (FIG. 6) and Mal (FIG. 7) provides a rationale for these inhibitory effects. In addition A46R was able to block MyD88-independent pathways (FIGS. 10 and 13) by associating with TRIF (FIG. 11). A46R was shown to be expressed early on in cells infected with VV (FIG. 14). Furthermore, a deletion mutant VV lacking the A46R gene was shown to be attenuated compared to wild type and revertant controls in vivo (FIG. 15), indicating the importance of A46R in viral virulence.

There is intense interest in the IL-1R/TLR family at present, given its emerging central importance in the innate immune response to diverse pathogens (Akira, S., Takeda, K & Kaisho, T. Nature Immunol. 2, 675-680 (2001)). During the course of viral infection the body mounts several lines of host defense involving constituents of the IL-1R/TLR superfamily. The cytokines IL-1 and IL-18 are key regulators of the innate and adaptive immune response to viral infection. In particular IL-1 is antagonized by the production of a soluble IL-1 binding protein (B15R) by VV (Alcami, A. & Smith, G. L. Cell 71, 153-167 (1992). L-18 is a potent inducer of IFN-, and administration of 118 has been shown to elicit antiviral effects in VV-infected mice (Tanaka-Kataoka, M. et al. Cytokine 11, 593-599 (1999)). Recent work has suggested that TLR3, TLR4 and TLR7 are crucial mediators of an innate immune response to viral infection (Kurt-Jones, E. A. et al. Nature Immunol. 1, 398-401 (2000); Rassa, J. C., Meyers, J. L., Zhang, Y., Kudaravalli, R. & Ross, S. Proc. Natl. Acad. Sci. USA 99, 2281-2286 (2002), Alexopoulou, L., Czopik-Holt, A., Medzhitov, R. & Flavell, R. Nature 413, 696-712 (2001) and Hemmi, H. et al. Nature Immunol. 3, 196-200 (2002)). Furthermore, TLR2 and TLR9 have also been implicated in responding to some viruses (Lund, J. et al J. Exp. Med. 198, 513-520 (2003); Compton, T. et al J. Virol. 77, 4588-4596 (2003). It is possible that other TLRs also have a role in responding to viral infection. The TLR family is therefore important in anti-viral host defense. Viral mechanisms to antagonise this family would have valuable therapeutic potential.

In the present invention the VV protein A46R has been found to be an IL-1R/TLR inhibitor. Deletion of A46R from VV causes the virus to be attenuated in a murine model of infection (FIG. 15). These results further support the emerging role of TLRs in the host response to viral infection.

A46R is very similar to the TIR domain and has wide-ranging effects on IL-1R/TLR signalling. The inhibitory data suggests that A46R blocks IL-1R/TLR signalling close to the receptors, before the NFκB and MAP kinase pathways bifurcate, probably by disrupting TIR-dependent interactions necessary for signalling. One important target for A46R is likely to be MyD88, since A46R can be co-immunoprecipitated with MyD88 (FIG. 6a), while GST-A46R can pull MyD88 out of a cell lysate (FIG. 6b), probably due to an association between the A46R and MyD88 TIR domains. This is the first demonstration of a viral protein targeting MyD88. The fact that A46R can also target Mal (as assessed by co-immunoprecipitation and GST pull-down, (FIG. 7) is consistent with the fact that TLR4 inhibition by A46R is particularly potent (compare FIG. 3 to FIG. 2).

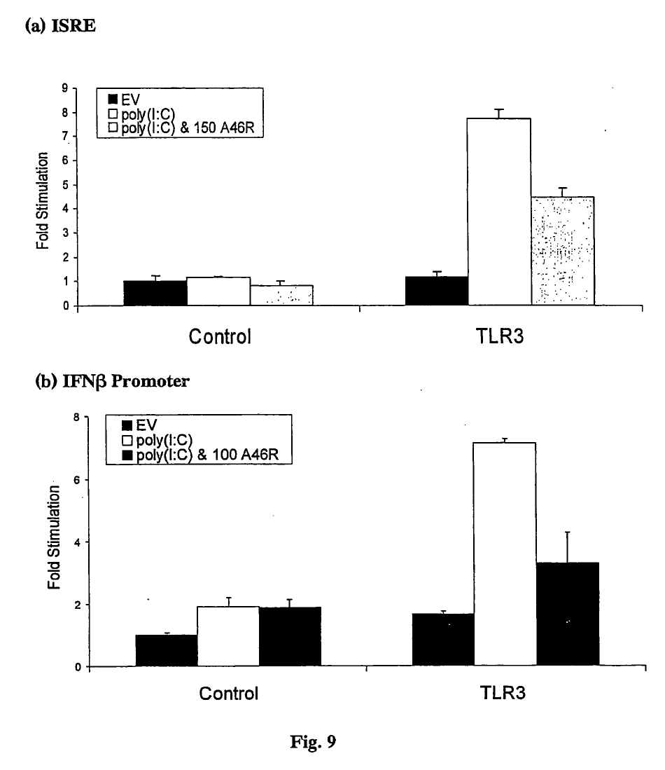

In addition to targeting MyD88- and Mal-dependent signalling, A46R was also found to block MyD88-independent TLR signalling events, such as those emanating from TLR3. FIG. 9 shows that both Interferon-Stimulated Response Element (ISRE) and Interferon-β (IFNβ) promoter induction by LR3 was potently blocked by A46R. This indicated that A46R might also target the adapter TRIF, since this has been shown to be responsible for most if not all TLR3-dependent. FIG. 10 shows that A46R could co-immunoprecipitate TRIF and also that GST-A46R was capable of pulling down TRIF.

Thus A46R, by nature of its similarity to the TIR domain, can target three key adapters involved in signalling by the IL-1/TLR superfamily, thus allowing A46R to antagonise a huge array of signalling pathways. The targeting of TRIF is particularly interesting, since it has recently been shown that TRIF controls the IFN-dependent, and thus anti-viral arm, of TLR3 and TLR4 signalling. When mice in which the TRIF gene was mutated were infected with cytomegalovirus, no IFN α/β was detected in the serum, although a robust IFN response was evident in control mice (Hoebe, K. et al Nature doi:10.1038/nature01889 (2003). Further, in macrophages in which 1RI is disrupted, vaccinia virus was able to replicate to a higher titre, compared to in control cells (Hoebe, K. et al Natuare doi:10.1038/nature01889 (2003)). This demonstrates a direct role for TRIF in containing viral infections, and hence makes it an important target for viral immune evasion strategies.

From the profile of expression of A46R in infected cells it was shown that the protein is expressed quite early in infection, compared to D8L, a known late expressed VV protein (FIG. 13a). This would be consistent with a role for A46R in suppressing IL-1R/TLR signalling in infected cells, since these receptors are generally involved in the triggering of host immune responses in the early phases of infection.

The present invention relates to a recombinant vaccinia virus in which the gene sequence of A46R is deleted. FIG. 13b confirms that the mutant virus does not express the A46R protein, while a revertant virus (in which the A46R gene is inserted into the virus deletion mutant lacking A46R) does. Deletion of A46R did not affect the replication of the virus in cell culture (FIGS. 13c and d). However, the absence of A46R led to an attenuation of the virus, in that when mice were infected intranasally, the deletion mutant caused a reduction in the weight loss induced in the animals, compared to wild type and revertant virus (FIG. 14).

Live vaccinia virus is currently used as the vaccine to immunise against and eradicate smallpox. However there is a need to develop more effective and safer smallpox vaccines due to the threat of bioterrorism. It is possible to engineer recombinant vaccinia viruses in which vaccinia genes are deleted or altered. Deletion or alteration of vaccinia virus genes involved in modulating the host immune response can alter the immunogenicity and safety of a vaccinia virus for use as a vaccine against smallpox or other orthopoxviruses, or for the development of recombinant vaccinia viruses as vaccines against other infectious diseases and cancer. Such recombinant vaccinia viruses can be engineered in which genes derived from other organisms are inserted (Macket, M. & Smith, G. L. J. Gen. Virol. 67, 2067-2082 (1986)). The recombinant viruses retain their infectivity and express any inserted genes during the normal replicative cycle of the virus. Immunisation of animals with recombinant viruses containing foreign genes has resulted in specific immune responses against the protein(s) expressed by the vaccinia virus, including those protein(s) expressed by the foreign gene(s) and in several cases has conferred protection against the pathogenic organism from which the foreign gene was derived. Recombinant vaccinia viruses have, therefore, potential application as new live vaccines in human or veterinary medicine.

The present invention also relates to a vaccinia virus wherein 93.5% of the nucleotide sequence encoding A46R is deleted. Alteration or deletion of A46R from the vaccinia genome may increase virus safety and immunogenicity. Such a virus or a derivative virus expressing one or more foreign antigens may have application as an improved vaccine against smallpox or other orthopoxvirses, or for the application of recombinant vaccinia viruses as vaccines against other infectious diseases and cancer.

The examples presented are illustrative only and various changes and modifications within the scope of the present invention will be apparent to those skilled in the art.

### EXAMPLES

Cell Culture. HEK 293, HEK 293T and RAW 264.7 cells were cultured in Dulbecco's modified Eagle's medium (DMEM) with 10% fetal bovine serum (FBS), supplemented with 100 units/ml penicillin, 100 mg/ml streptomycin, and 2 mM L-glutamine.

Expression Plasmids. The chimeric TLR receptor CD4-TLR4, composed of the extracellular domain of CD4 fused to the transmembrane domain and cytosolic tail of TLR4 was a gift from R. Medzhitov, (Yale University, New Haven, Conn.). AU1-MyD88 expression vector was a gift from M. Muzio (Muzio, M., Ni, J., Feng, P. & Dixit, V. M. Science 278, 1612-1615 (1997)). The mammalian expression vector pRK5 was kindly provided by Tularik Inc. (San Francisco, Calif.). Flag-TRIF was from S. Akira (Research Institute for Microbial Diseases, Osaka University, Japan). A46R, Flag-A46R and HA-Mal expression plasmids have been previously described (Bowie, A. et al. Proc. Natl Acad Sci. USA 97, 10162-10167 (2000); Fitzgerald, K. A. et al Nature 413, 783 (2001)).

The name A46R is based on the standard VV nomenclature of the Copenhagen strain (Goebel, S. J et al, Virology 179, 247-266 (1990)). A46R was cloned from the laboratory VV strain WR where it was previously called SalF9R (Smith, G. L et al J. Gen. Virol, 72 1349-1376 (1991); Goebel, S. J et al, Virology 179, 247-266 (1990)) into the mammalian expression vector pRK5. Any other suitable mammalian expression vector such as pcDNA3.1 (available from Invitrogen) or pEF-BOS (Mizushima et al Nucleic Acids Res. 18, 5322 (1990)) for example may also be used.

The A46R ORF was cloned by PCR amplification from WR DNA with primers incorporating restriction sites for EcoRI upstream and HindIII downstream of the ORF. The primers used were 5′-CGTGAATTCCGAGAATGGCGTTTGA (sense) and 5-CGGAAGCTTTTATACATCCGTTTCCT (antisense). The restriction sites and start and stop codons are underlined. The resulting EcoRI-HindIII fragment was ligated into the multiple cloning site of the mammalian expression vector pRK5. For immunoblot analysis, an epitope-tagged A46R expression vector was constructed, employing the same strategy, except that the 8-amino acid Flag coding sequences was inserted into the antisense primer 5′ of the stop codon.

Antibodies. Polyclonal antibodies were raised against a purified, bacterially expressed glutathione S-transferase (GST) fusion of A46R, encoded by a plasmid synthesised by inserting full length A46R downstream of GST in the bacterial expression vector GEX4T2. Other antibodies used were anti-AU1 monoclonal antibody (BabCO), anti-HA polyclonal antibody (Y-11, Santa Cruz Biotechnology) and anti-flag M2 monoclonal antibody (Sigma)

Luciferase reporter gene assays. HEK 293 cells (2×10⁴ cells per well) or RAW 264.7 cells (4×10⁴ cells per well) were seeded into 96-well plates and transfected the next day with expression vector, and reporter plasmids. GeneJuice™ (Novagen) was used for transient transfections, according to the manufacturer's instructions. For experiments involving NFκB, ISRE or IFNβ promoter, 60 ng of κB-luciferase reporter gene, ISRE-luciferase reporter gene (Stratagene) or INFβ promoter luciferase reporter (a gift from Prof. Taniguchi, University of Tokyo) respectively were used as previously described (Fitzgerald, K. A. et al. Nature 413, 78-83 (2001)). For MAP kinase reporter assays the Stratagene Pathdetect System™ was used whereby c-jun (2 ng) or Elk1 (5 ng) Gal4 fusion vectors were used in combination with 80 ng pFR-luciferase reporter to measure JNK and ERK activation respectively (Fitzgerald, K. A. et al. Nature 413, 7843 (2001)). For p65 or IRF3 transactivation assay, a p65-Gal4 or IRF3-Gal4 fusion vector was used in combination with pFR-luciferase reporter (Jefferies, C., et al. Mol. Cell. Biol. 21, 454-44552 (2001)). In all cases 40 ng of Renilla-luciferase internal control (Promega) was used. The total amount of DNA per transfection was kept constant at 200 ng by addition of pcDNA3.1 (Stratagene). After 24 h cells were harvested into passive lysis buffer (Promega) and reporter gene activity was measured in a luminometer. Data are expressed as mean fold induction±s.d. relative to control levels, for a representative experiment from a minimum of three separate experiments, each performed in triplicate.

Immunoprecipitation and GST-Pulldown Assays. HEK 293T cells were seeded into 100 mm dishes (1.5×10⁶) 24 hrs prior to transfection. Transfections were carried out using GeneJuice™ (Novagen) according to manufacturers instructions. Four g of each construct was transfected. Where only one construct was expressed the total amount of DNA (8 g) was kept constant by supplementation with vector DNA. Cells were harvested 24 hrs post transfection in 750 l of lysis buffer (50 mM HEPES, pH 7.5, 100 mM NaCl, 1 mM EDTA, 10% glycerol, 0.5% NP40 containing 1 mM PMSF and protease inhibitor cocktail ( 1/100) (Sigma), and 1 mM sodium orthovanadate). For immunoprecipitation the indicated antibodies were precoupled to either protein A sepharose or protein G sepharose (anti-AU1) for 1 hr at 4° C., washed, and then incubated with the cell lysates for 2 hrs at 4° C. The immune complexes were washed twice with lysis buffer and once with lysis buffer without NP40 and glycerol. Associated proteins were eluted from the beads by boiling in 35 l of 3×SPB (final concentrations in sample: 62.5-mM Tris, 2% (w/v) SDS, 10% v/v glycerol, 0.1% (w/v) bromophenol blue)). The immune complexes were analyzed by SDS PAGE. 30 l of the immune complex was immunoblotted for co-precipitating protein and the remaining 5 l was blotted directly for the protein directly recognised by the immunoprecipitating antibody. For immunoblotting, primary antibodies were detected using horseradish peroxidase conjugated secondary antibodies, followed by enhanced chemiluminescence (Amersham). For GST-pulldown experiments, a similar transfection protocol was employed, except that cells were transfected with 8 μg of plasmid expressing a TIR adapter. GST-pulldown assays were performed using recombinant GST-A46R fusion protein (prepared and purified using standard techniques) coupled to GSH-sepharose. Lysates prepared as described above were incubated for two hours with GST-A46R, washed three times as above, and subjected to SDS-PAGE and immunoblotting.

Plaque Assays. Aliquots from vaccinia virus stocks (WR strain) were frozen and thawed 3 times, sonicated and serial dilutions were made in 2.5% FBS DMEM. Three of these dilutions were inoculated in duplicate onto confluent monolayers of BSC-1 cells in 6-well plates (0.5 ml of each dilution per well). After infection for 90 min at 37 C, the inoculum was aspirated, cells were overlaid with 2 ml of 1.5% carboxymethylcellulose (CMC) in 2.5% FBS DMEM and incubated for 2 days at 37 C. The semi-solid overlay was aspirated, cells were washed briefly with PBS and stained with 0.1% (w/v) crystal violet in 15% ethanol. After rinsing with water, the plate was air-dried and the number of plaques was determined.

### Example 1

### A46R Inhibition of Multiple Signals Induced by the IL-1R/TLR Superfamily

### (i) A46R Inhibits Multiple IL-1-Dependent Signals

A46R has been shown to block IL-1 induced NFkB activation, while not affecting TNF (Bowie, A. et al. Proc. Natl Acad. Sci. USA 97, 10162-10167 (2000)). To determine what other signals activated by IL-1 would also be blocked by A46R HEK 293 cells (2×10⁴ cells per well) were transfected with a % B-luciferase reporter gene and Renilla-luciferase internal control as described above. Six hours prior to harvesting, cells were stimulated with 100 ng/ml IL-1. Cells were harvested 24 h after transfection, and the reporter gene activity was measured. Data is expressed as mean fold induction±s.d. relative to control levels, for a representative experiment from a minimum of three separate experiments, each performed in triplicate. FIG. 2a shows that as the amount of expression vector encoding A46R transfected into cells increased, there was a modest dose-dependent inhibition of IL-1 induced NFκB activation, as previously shown (Bowie, A. et al. Proc. Natl Acad. Sci USA 97, 10162-10167 (2000)). A46R was found to block other signals induced by IL-1. A single dose of A46R cDNA transfected into cells was capable of blocking the ability of the transactivating subunit of NFκB, p65 to activate a reporter gene (FIG. 2b), JNK activation (FIG. 2c), and ERK activation (FIG. 2d), respectively

### (ii) A46R Inhibits Multiple TLR4-Dependent Signals

As A46R has a putative TIR domain we determined whether other TIR-dependent signals were sensitive to inhibition by examining the TLR4 pathway. Chimeric versions of the TLRs, comprising the murine CD4 extracellular domain fused to the cytoplasmic domain of a given human TLR have proved useful in probing TLR signalling pathways (Hayashi, P. et al. Nature 410, 1099-1103 (2001); Ozinsky, A. et al. Proc. Natl. Acad. Sci. USA 97, 13766-13771 (2000); Medzhitov, R., Preston-Hurlburt, P. & Janeway, C. A. Jr. Nature 388, 394-397 (1997)). The extracellular domain of CD4 promotes homodimerisation of the molecules. Chimeras composed of the extracellular domain of CD4 fused to the intracellular domain of TLR4 are constitutively active, in that overexpression of CD4-TLR4 induces NF B activation and gene induction (Medzhitov, R., Preston-Hurlburt, P. & Janeway, C. A. Jr. Nature 388, 394-397 (1997)). In the present invention HEK 293 cells (2×10⁴ cells per well) were transfected with a Renilla-luciferase internal control and either κB-luciferase construct (FIG. 3a), or plasmids encoding Gal4 fused to p65 (for p65, (b)), Elk1 (for ERK1/2, (c)) or CHOP (for p38, (d)) in the presence or absence of 50 ng CD4-TLR4, together with increasing amounts of A46R cDNA. Cells were harvested 24 hours after transfection, and the reporter gene activity was measured. Data are expressed as mean fold induction±s.d. relative to control levels, for a representative experiment from a minimum of three separate experiments, each performed in triplicate. Cells transfected with empty vector were used as control.

NκFB activation was measured as shown in FIG. 3a Overexpression of CD4-TLR4 in BE293 cells led to induction of the NFκB-dependent reporter gene (FIG. 3a) which was inhibited dose-dependently by A46R. The effect of A46R on TLR4 appears to be more potent than its effects on IL-1 (compare FIG. 2a and FIG. 3a). A46R was found to block other signals induced by CD4-TLR4. A single dose of A46R cDNA transfected into cells was capable of blocking the ability of the transactivating subunit of NFκB, p65, to activate a reporter gene (FIG. 3b), ERK activation (FIG. 3c), and p38 activation (FIG. 3d), respectively.

(iii) A46R Inhibits TLR Ligand-Induced NFκB Activation in Murine Macrophages

Murine macrophage RAW264.7 cells were transfected with an NKκB luciferase construct and a Renilla-luciferase internal control, together with empty vector (EV) or 100 ng cDNA encoding A46R. Cells were stimulated for 6 hours with the TLR agonists 1 M R-848 (TLR7), 1000 ng/ml LPS (TLR4) or 250 ng/ml flagellin (TLR5) before harvesting. 24 h after transfection cells were harvested, and the reporter gene activity was measured. Data are expressed as mean fold induction±s.d. relative to control levels, single experiment, performed in triplicate.

A46R inhibited NKκB activation induced by R-848, LPS, and flagellin. As in the case of CD4-TLR4 in 293 cells (FIG. 3a), TLR4 (LPS) activation was again particularly sensitive to A46R inhibition. R-848 induced NKκB activation was also very strongly inhibited by A46R.

These results show that A46R is capable of blocking multiple signals emanating from the IL-1R and TLRs. The fact that A46R has a putative TIR domain suggests that it might block these TIR-dependent pathways by disrupting the TIR receptor interactions necessary for signalling. Thus A46R probably acts close to the receptors, before signal bifurcation.

### Example 2

### A46R Associates with MyD88 and Mal, and can Block Signals Induced by MyD88 and Mal Overexpression

(i) Association of A46R with MyD88.

The activation of NFκB by IL-1R/TLR family members is mediated by a common set of signalling molecules. The ability of A46R to inhibit NFκB activation induced by IL-1 TLR4, TLR5 and TLR7 suggested that its effects may be due to its interaction with a molecule whose function is critical to signalling by all these receptors. MyD88 was a likely target, given its role in these pathways, and the fact that it has a TIR domain. Therefore the ability of A46R to interact with MyD88 was examined.

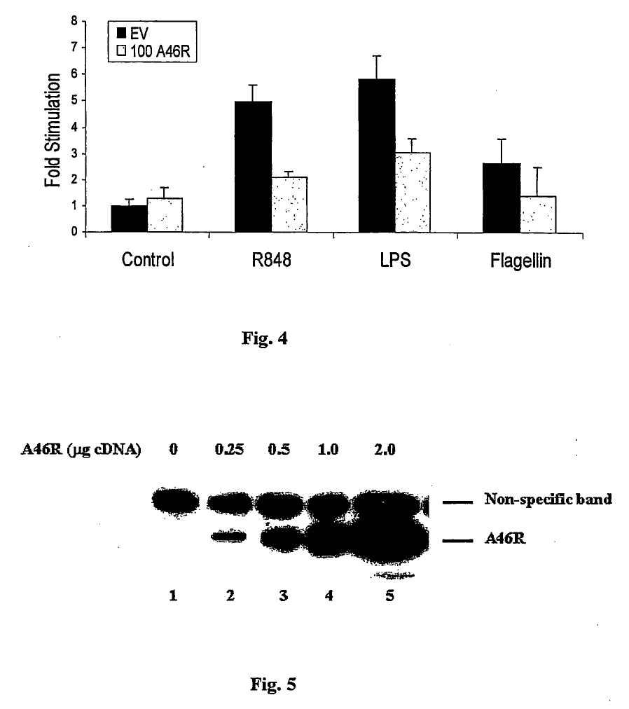

Firstly, dose-dependent expression of A46R was demonstrated in SK 293T cells, HEK 293T cells were seeded at 1×10⁵ cells/ml in 6 well plates 24 h prior to transfection. Transfections were carried out using GeneJuice™ (Novagen) according to manufacturers instructions. Increasing amounts of a plasmid vector encoding A46R was transfected, as indicated in FIG. 5. The total amount of DNA (2 g) was kept constant by supplementation with vector DNA. Cells were harvested 24 h post transfection and resolved by SDS-PAGE. The blot was probed with an antibody specific for A46R and FIG. 5 shows that a clear dose-dependent pattern of expression was observed, at the correct molecular mass for A46R (near 26 kD).

In order to test the ability of A46R to associate with MyD88, HEK 293T cells were transfected with plasmids encoding A46R and AU1-MyD88, and co-immunoprecipitation performed, as described above. The results are shown in FIG. 6a, where lanes 1-3 correspond to lysates directly blotted for expression of MyD88, lanes 46 correspond to lysates immunoprecipitated with anti-A46R antibody and blotted for the presence of MyD88, while lanes 7-9 correspond to immunoprecipitation using anti-AU1 antibody directed towards AU1-MyD88. Therefore, the appearance of a band in lane 6 that is not detected in lanes 4 or 5 is indicative of an interaction between A46R and MyD88. This is clearly evident in FIG. 6a, demonstrating that A46R immunoprecipitates in complex with MyD88. Association between A46R and MyD88 was also shown by GST-pulldown, where GST-A46R could pull MyD88 out of a cell lysate (FIG. 6b). Lane 1 corresponds to lysate directly blotted for expression of MyD88, lane 2 corresponds to lysate incubated with GST and lane 3 corresponds to lysate incubated with GST-A46R.

(ii) Association of A46R with Mal

The ability of A46R to associate with Mal, another TIR domain containing adaptor molecule, that has a specific role in TLR2 and TLR4 signalling was also tested. A46R was expressed in HEK 293T cells along with Flag-tagged Mal. To isolate complexes, immunoprecipitations were carried out using antibodies directed against Flag or A46R. The results are shown in FIG. 7a, where lanes 1-3 correspond to lysates directly blotted for expression of A46R, lanes 4-6 correspond to lysates immunoprecipitated with Flag antibody and blotted for the presence of the A46R, while lanes 7-9 correspond to immunoprecipitation using A46R antibody. Therefore, the appearance of a band in lane 6 that is not detected in lanes 4 or 5 is indicative of an interaction between A46R and Mal. Similar to MyD88, Mal could also be found associated with A46R, since Mal immunoprecipitated in complex with A46R (FIG. 7a, lane 6). This result was confirmed by GST-pulldown, where GST-A46R pulled HA-Mal out of a cell lysate (FIG. 7b, lane 3).

(iii) Inhibition by A46R of Signals Induced by Either MyD88 or Mal Overexpression

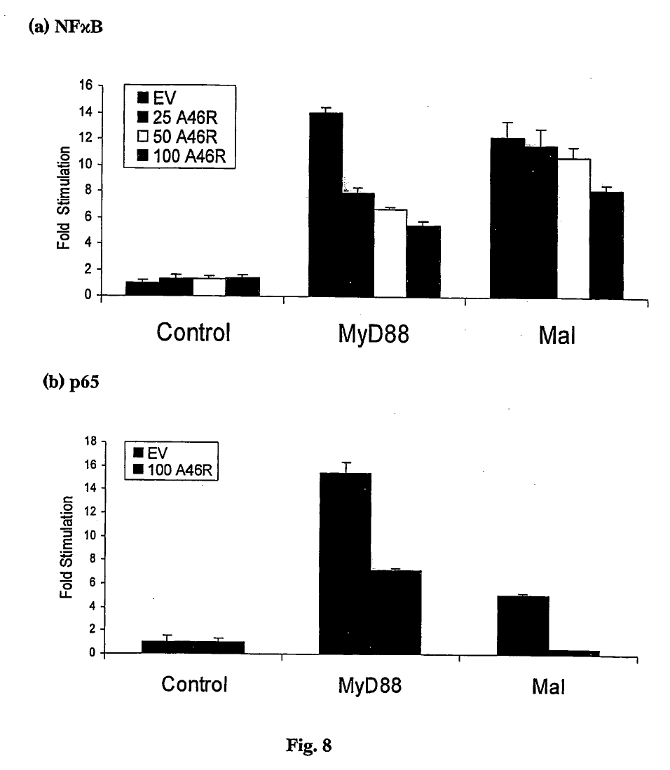

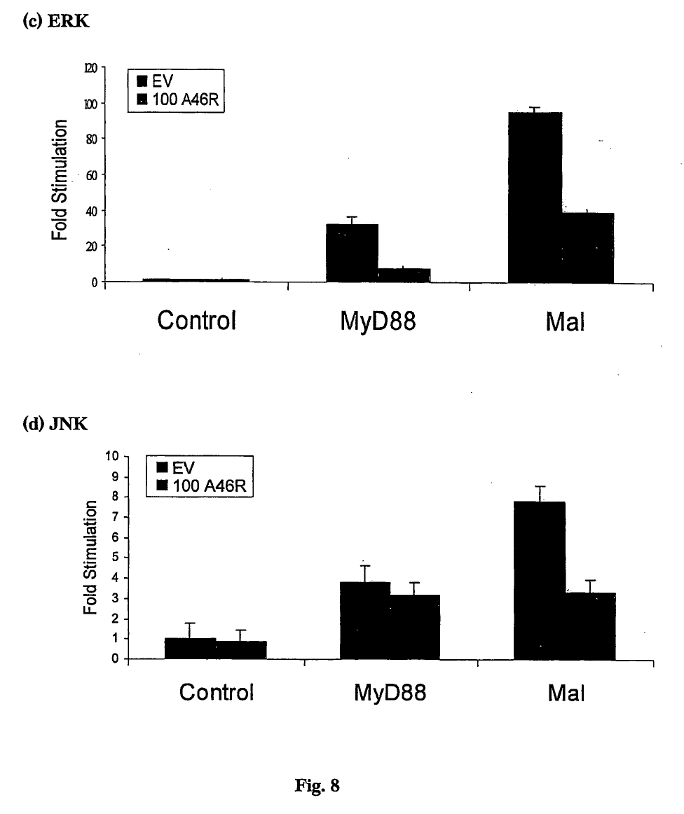

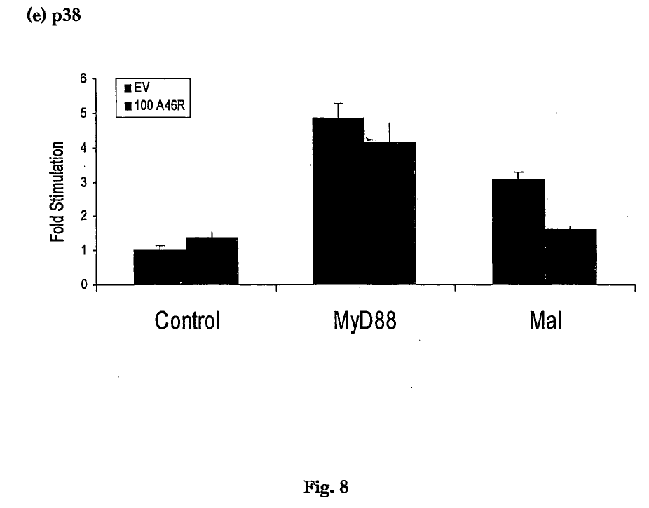

The effect of A46R on signals induced by ectopic expression of MyD88 or Mal was determined in order to test more directly the implications of A46R associating with both MyD88 and Mal. HEK 293 cells (2×10⁴ cells per well) were transfected with different amounts of cDNAs encoding either MyD88 or Mal, in the presence or absence of A46R, together with a Renilla-luciferase internal control and either κB-luciferase construct (FIG. 8a), or plasmids encoding Gal4 fused to p65 (for p65, (FIG. 8b)), Elk1 (for ERK1/2, (FIG. 8c)), c-JUN (for JNK, (FIG. 8d)) or CHOP (for p38, (FIG. 8e)). Cells were harvested 24 hours after transfection, and the reporter gene activity was measured. Data are expressed as the mean fold induction±s.d. relative to control levels, for a representative experiment from a minimum of three separate experiments, each performed in triplicate. Cells transfected with empty vector were used as control. The amounts of cDNA used were 2 ng Mal or 5 ng MyD88 for NF B reporter gene assay (a), 50 ng MyD88 or Mal for p65 transactivation assay (b), 25 ng MyD88 or Mal for JNK assay (d), and 10 ng MyD88 or 25 ng Mal for ERK (c) and p38 (e) assays. These amounts of cDNA were determined as being optimal for activation of the different reporters in previous experiments.

Consistent with the targeting of MyD88 and Mal by A46R, both MyD88- and Mal-mediated NF B, p65 and ERK signals are inhibited very potently by A46R (FIG. 8a to c). However, A46R inhibits MyD88 mediated JNK and p38 signals only slightly, when compared to the very strong inhibition of Mal mediated JNK and p38 signals by A46R (FIGS. 8d and e).

Thus the ability of A46R to antagonise multiple signals induced by IL-1 and TLRs is likely due to its ability to associate with MyD88 and Mal. The functional consequences of these associations is that A46R can inhibit signals emanating from these adaptors.

### Example 3

### A46R Blocks MyD88-Independent Pathways by Associating with TRIF

A46R was also capable of antagonising TLR signalling pathways known to be independent of MyD88 and Mal. One important example of such a pathway is the activation of ISRE promoter elements and subsequent induction of IFNβ by poly(I:C) via TLR3. In order to test the effect of A46R on this pathway, HEK 293 cells (2×10⁴ cells per well) were transfected with 0.5 ng TLR3 in the presence or absence of A46R, with an ISRE-luciferase construct (FIG. 9a), or an IFNβ promoter reporter plasmid (b) and Renilla-luciferase internal control. Cells were harvested at 24 hours, 6 hours after stimulation with 25 μg/ml poly(I:C), and the reporter gene activity was measured Data are expressed as mean fold induction±s.d. relative to control levels, for a representative experiment from a minimum of three separate experiments, each performed in triplicate. FIG. 9 shows that A46R potently blocked both ISRE (a) and IFNβ promoter (b) activation, suggesting that A46R can also target the MyD88-independent, IRF3-dependent pathway. This pathway has recently been shown to be controlled by the TIR adapter TRIF (Hoebe, K. et al Nature doi:10.1038/nature01889 (2003); Yamamoto, M. et al Science doi:10.1126/science.1087262 (2003)). The same immunoprecipitation and GST-pulldown approach used for MyD88 and Mal was employed to test whether A46R could associate with TRIF, in addition to MyD88 and Mal. FIG. 10 shows that A46R was indeed able to associate with TRIF in both assays (FIG. 10a, lane 6 and FIG. 10b, lane 3).

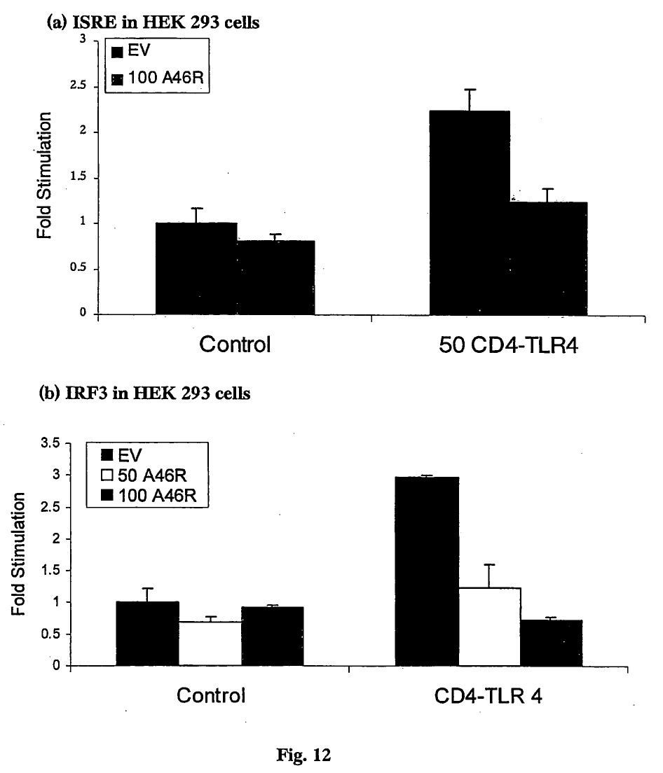

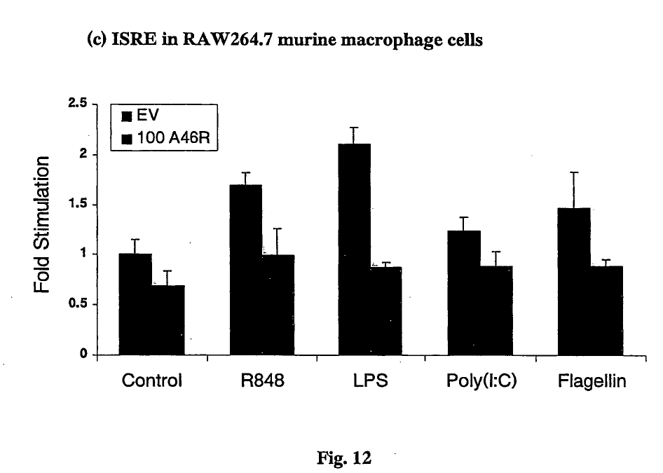

Thus A46R can target TRIF-dependent signalling by association with this TIR adaptor. This was confirmed in other experiments where activation of either NFκB or induction of the IFNβ promoter by ectopic expression of TRIF was blocked by A46R (FIGS. 11a and b respectively). Furthermore, TLR4-induced ISRE or IRF3, which are also dependent on TRIF, were also potently inhibited (FIGS. 12a and b). Finally, FIG. 12c shows that TLR ligand induced ISRE induction in murine macrophages by LPS, and other TLR agonists, is also sensitive to A46R inhibition.

Overall these results show that by targeting three different TIR adapters, A46R can inhibit multiple IL-1 and TLR induced signals.

### Example 4

### Comparison of a VV Deletion Mutant Lacking A46R Gene with Wild Type and Revertant Viruses

A mutant virus lacking the A46R gene was constructed in order to determine what contribution A46R might make to W virulence. A revertant virus, in which the A46R gene was re-inserted into the mutant, was also constructed.

(i) Expression of A46R in Cells Infected with Wild Type Virus, and Characterisation of VV Deletion Mutant Lacking A46R

The expression profile of A46R in cells infected with wild-type virus was determined. BSC-1 cells were mock infected or infected with W WR at 10 pfu/cell in the absence or presence of 40 mg/ml cytosine arabinoside (an inhibitor of DNA synthesis). At various times p.i., cells were harvested, and extracts were prepared, separated by SDS-PAGE and analysed by immunoblotting. Blots were detected with anti-A46R antibody (diluted 1:1000) and anti-D8L antibody (diluted 1:1000). D8L is known to be expressed late during VV infection. Bound IgG was detected with HRP-conjugates goat anti-rabbit IgG antibody (diluted 1:2500) and ECL reagents (Amersham) and blots were exposed to X-OMAT film.

FIG. 13a shows that in comparison to D8L, which was only detected at 24 h p.i., A46R could be seen after just 6 h. The level of protein continued to accumulate up to 24 h. This profile of expression of A46R in infected cells shows that the protein is expressed quite early in infection, and rapidly accumulates, compared to D8L, a known late expressed VV protein. This is consistent with a role for A46R in suppressing IL-1R/TLR signalling in infected cells, since these receptors are generally involved in the triggering of host immune responses in the early phases of infection.

The role of A46R in the VV life cycle was investigated by the construction of a deletion mutant lacking the A46R gene and by the comparison with wild type and revertant controls. A VV mutant lacking 93.5% of the A46R gene (v A46R) was constructed by transient dominant selection (Falkner, F. G. & Moss, B. (1991) J. Virol. 64, 3108-3111). A plaque purified wild type virus (vWT-A46R) and a revertant virus (vA46R-RV) in which the A46R gene was reinserted at its natural locus were also isolated. FIG. 13b confirms that no A46R was expressed in cells infected with the v A46R virus, while those infected with vWT-A46R and vA46R-RV did express the protein. BSC-1 cells were infected with vWT-A46R, vA46 R and vA46R-RV at 10 pfu/cell. Cells were harvested 8 h p.i. and extracts were prepared, separated by SDS-PAGE and analysed by immunoblotting as before.

The loss of the A46R gene did not affect the replication of the virus in cell culture, as shown in FIGS. 13c and d. FIG. 13c is a single step growth analysis of recombinant viruses. BSC-1 cells were infected with the indicated viruses at 10 pfu/cell. After 24 h, the virus present in the clarified supernatants and cells were determined by plaque assay in duplicate. Results show the mean of duplicate experiments. As can be seen, there is no difference between the viral titre for vWT-A46R, vA4 6 R and vA46R-RV. FIG. 13d shows multi-step growth curves for the recombinant viruses. BSC-1 cells were infected with the indicated viruses at 0.01 pfu/cell. At various times post-infection, virus was harvested by scraping cells into the culture supernatants. Samples were frozen and thawed 3 times and sonicated. Total virus levels were determined by plaque assay on BSC-1 cells. Results show the mean of duplicate experiments. vWT-A46R, v A 46R and vA46R-RV display the same growth characteristics.

The deletion of A46R therefore does not affect the growth or replication of VV in cell culture.

(ii) Deletion of A46R Gene from VV Attenuates the Virus

The virulence of the virus was examined in a mouse intranasal model. Groups of five female, 6-week old Balb/c mice were anaesthetized and inoculated with 10⁴ pfu of VV in 20 μl of phosphate-buffered saline (PBS). A control group was mock infected with PBS. Each day the weights of the animals was measured as described previously (Alcami, A. & Smith, G. L. (1992) Cell 71, 153-167). Data are presented as the mean weight of each group of animals compared to the mean weight of the same group on day 0. As can be seen in FIG. 14, the deletion mutant caused reduced weight loss in mice, compared to wild-type and revertant viruses.

Thus the A46R protein contributes to virus virulence and this is likely to be due to the inhibition of IL-1/TLR signalling.

These results demonstrate that A46R from VV is able to inhibit IL-1R/TLR-induced intracellular signalling, by associating with a TIR adapter-containing complexes. The ability of A46R to disrupt TLR signalling has relevance to VV virulence, since deletion of A46R attenuates the virus.

In this specification some references have been included which were published after the priority date of the application. These are included for the reader's assistance only.

The invention is not limited to the embodiments hereinbefore described which may be varied in detail.

1-12. (canceled)

13. A method of inhibiting IL1R/TLR superfamily signalling comprising the step of:
administering to a subject an effective amount of vaccinia virus A46R protein or a functional peptide, peptidomimetic, fragment or derivative thereof, or a DNA vector capable of expressing vaccinia virus A46R protein or the functional peptide, peptiomimetic, fragment or derivative thereof.

14-15. (canceled)

16. A method for the modulation and/or inhibition of IL1R/TLR superfamily signalling comprising the step of:
administering to a subject an effective amount of vaccinia virus A46R protein, or a functional peptide, peptidomimetic, fragment or derivative thereof, or a DNA vector expressing vaccinia virus A46R protein or the functional peptide, peptiomimetic, fragment or derivative thereof.

17. The method as claimed in claim 16 wherein IL1R/TLR superfamily-induced NFκB activation is modulated and/or inhibited.

18. The method as claimed in claim 16 wherein IL1R/TLR superfamily-induced MAP kinase activation is modulated and/or inhibited.

19. The method as claimed in claim 16 wherein TLR induced IRF3 activation is modulated and/or inhibited.

20. The method as claimed claim 16 wherein Toll-like receptor proteins are inhibited by the vaccinia virus A46R protein or functional peptide, peptiomimetic, fragment or derivative thereof.

21. The method as claimed in claim 16 wherein NFκB activity or MAP kinase activation is modulated and/or inhibited by interaction of the vaccinia virus A46R protein or functional peptide peptiomimetic, fragment or derivative thereof with MyD88.

22. The method as claimed in claim 16 wherein NFκB activity or MAP kinase activation is modulated and/or inhibited by interaction of the vaccinia virus A46R protein or functional peptide peptiomimetic, fragment or derivative thereof with Mal.

23. The method as claimed in claim 16 wherein MyD88- and/or Mal-dependent signalling is inhibited by the vaccinia virus A46R protein or functional peptide, peptiomimetic, fragment or derivative thereof.

24. The method as claimed in claim 16 wherein IRF3 or NFκB activity is modulated and/or inhibited by interaction of the vaccinia virus A46R protein or functional peptide, peptiomimetic, fragment or derivative thereof with TRIF.

25. The method as claimed in claim 16 wherein TRIF-dependent signaling is inhibited by the vaccinia virus A46R protein or functional peptide, peptiomimetic, fragment or derivative thereof.

26-37. (canceled)
